(19) United States
(12) Patent Application Publication
(10) Pub. No.: US 2018/0208809 A1
(43) Pub. Date: Jul. 26, 2018

(54) ELASTIC ADHESIVE COMPOSITION AND AN ELASTIC COMPOSITE MADE WITH THE SAME

(71) Applicant: H.B. Fuller Company, St. Paul, MN (US)

(72) Inventors: Kevin P. Davis, Woodbury, MN (US); Pag Moua, St. Paul, MN (US); Tara Misra, Shoreview, MN (US); Sehban N. Ozair, Blaine, MN (US); Sophorn Pin, Stillwater, MN (US)

(21) Appl. No.: 15/878,048

(22) Filed: Jan. 23, 2018

Related U.S. Application Data
(60) Provisional application No. 62/450,636, filed on Jan. 26, 2017.

(51) Int. Cl. C09J 153/02; A61F 13/49

(52) U.S. Cl. CPC C09J 153/02; A61F 13/49011; A61F 13/49014

(57) ABSTRACT

This invention claims hot melt adhesive compositions that can be used to form elastic composites that are useful in disposable absorbent articles.

The hot melt adhesive compositions combine several desirable properties including low viscosity, low odor, a desirable retractive profile improved snap back) as demonstrated by low mechanical hysteresis values and acceptable adhesion. These properties make the hot melt adhesives well suited to forming elastic composites. The improved retractive profile further allows for the ability to apply a lower coat weight and still maintain the elastic properties.

This application claims the benefit of U.S. Provisional Application No. 62/450,636, filed Jan. 26, 2017, which is incorporated herein.

### BACKGROUND

Adhesives are often used to bond substrates together. In the area of industrial adhesives, hot melt adhesives are commonly used to bond together a wide variety of articles including disposable absorbent articles comprising non-woven substrates e.g. diapers, training paints, surgical garments, swim wear, absorbent underpants, adult incontinence products, sanitary napkins and medical dressings (e.g. wound care products).

There can be multiple hot melt adhesives used in the manufacture of a disposable absorbent article. For example, in the manufacture of a disposable diaper, hot melt adhesives are used for construction (e.g, bonding the backsheet to the nonwoven and optionally the absorbent pad), elastic attachment (e.g. bonding the elastic material to the backsheet in for example the leg or waist area), and for the core stabilization (e.g. applying an adhesive to the absorbent core to increase the strength of the core).

Hot melt adhesives can also be used to form elastic composites that are useful in disposable articles. Currently, elastic composites are often formed in a 5-layer configuration including the following layers: nonwoven, hot melt adhesive, elastic material, hot melt adhesive, nonwoven. The hot melt adhesive bonds the non adhesive elastic to the nonwoven to form a composite.

Alternately, a hot melt adhesive with elastic properties can replace both the elastic material and the adhesive layers to form a simplified 3-layer elastic composite that can impart stretch to various portions of the disposable article.

Styrene block copolymers (SBC) have been used as a polymer in such compositions.

It would be desirable to be able to formulate a low viscosity hot melt adhesive based on SBC, with a desirable retractive profile (i.e. improved snap back) so as to be useful in constructing a 3-layer elastic composite. It would further be useful if such properties could be obtained at a relatively lower coat weight.

### SUMMARY

Applicants have discovered hot melt adhesive compositions that combine several desirable properties including low viscosity, low odor, a desirable retractive profile (i.e. improved snap back) as demonstrated by low mechanical hysteresis values and acceptable adhesion. These properties make the hot melt adhesives well suited to forming elastic composites. The improved retractive profile further allows for the ability to apply a lower coat weight and still maintain the elastic properties.

In one embodiment, the invention includes an elastic composite including a first and second substrate; and a hot melt adhesive composition between the first and second substrates thereby bonding the first and second substrates to each other and providing elasticity in the bonded area, said hot melt adhesive composition including from about 45% to about 75% by weight of one or more styrene block copolymers having an unsaturated mid-block and an average styrene content of at least about 20%, less than 20% by weight of a tackifying agent, from about 10% to about 60% by weight of a plasticizer, the hot melt adhesive composition having a viscosity of no greater than about 20,000 cps at 177° C.

In one aspect, the elastic composite includes a hot melt adhesive composition having a Mechanical Hysteresis 50% strain (coat weight 60 gsm) 2nd cycle of no greater than 0.38. In another aspect, the hot melt adhesive composition includes one or more styrene block copolymers has an average dibiock content of no greater than about 10% by weight. In a different aspect, the hot melt adhesive composition includes one or more styrene block copolymers has an average styrene content of at least about 25% by weight. In one aspect, the hot melt adhesive composition includes at least one styrene block copolymer having an ASTM D 1238 (200° C., 5.0 kg) melt flow rate of at least about 100.

In one aspect, the elastic composite includes a hot melt adhesive composition including from about 2% to about 15% by weight of a tackifying agent. In a different aspect, the hot melt adhesive composition includes from about 3% to less than 10% by weight of a tackifying agent. In one aspect, the hot melt adhesive composition further includes a paraffin wax.

In a different aspect, the first and second substrates of the elastic composite are nonwoven. In another aspect, the nonwoven is carded and manufactured in a method selected from hydroentangied and thermally bonded. The still another aspect, the nonwoven is extensible to greater than 100% in the cross web direction.

In one aspect, the hot melt adhesive of the elastic composite is applied to the substrate using an applicator method selected from the group consisting of slot and non-contact coating. In a different embodiment, the hot melt adhesive is applied to the substrate using an applicator method selected from the group consisting of screen printing, spraying, comb shim slot and gravure roll.

The invention further includes a disposable article comprising any of the above elastic composites. In a different aspect, the disposable article is selected from the group consisting of diaper, adult incontenence product, feminine hygiene product and medical bandage. In another aspect, the elastic composite is used in an application selected from the group consisting of ear, waist band, belly band and side panel.

In one embodiment, the invention features a hot melt adhesive composition that can be used to create a bonded, elasticized region including from about 45% to about 75% by weight of one or more styrene block copolymers, the styrene block copolymers having unsaturated backbones, an average styrene content of at least about 25% by weight, and a diblock content of no greater than about 10% by weight, from about 5% to about 15% by weight of a tackifying agent and, from about 15% to about 45% by weight plasticizer, the adhesive composition having a viscosity of no greater than about 20,000 cps at 177° C.

In one aspect, the hot melt adhesive composition has a Mechanical Hysteresis 50% strain (coat weight 60 gsm) 2nd cycle of no greater than 0.38. In a different aspect, the hot melt adhesive composition viscosity of no greater than about 15,000 cps at 177° C. The hot melt adhesive composition includes at least one styrene block copolymer having an ASTM D 1238 (200° C., 5.0 kg) melt flow rate of at least about 150.

Other features and advantages will be apparent from the following description of the preferred embodiments and from the claims.

### DETAILED DESCRIPTION

Applicants have discovered hot melt adhesive compositions that can be used to form elastic composites that are useful in disposable articles (e.g. disposable absorbent articles). The compositions have a desirable retractive profile and a viscosity of no greater than about 20,000 cps at 177° C. The low viscosity makes it possible to apply the adhesive at a high line speed and target application to only those areas requiring elastic performance.

### Elastic Composite

The elastic composite can include a first substrate and a hot melt adhesive composition. The hot melt adhesive composition imparts elasticity to the first substrate.

Alternately, the elastic composite can include a first substrate, a second substrate and a hot melt adhesive composition. The hot melt adhesive composition is present between the first and second substrate, bonding the substrates to each other and providing elasticity in the bonded area. The substrates can be permanently bonded to each other.

At least one of the substrates is selected from the group consisting of nonwoven and polymer film.

Any nonwoven can be used. The nonwoven can be an elastic nonwoven (e.g. core and shell type). The nonwoven can contain fibers made from one or more polymers (e.g. PET (polyethylene terephthalate), PBT (polybutylene terphthalate), nylon, polypropylene and polyethylene), one or more natural fibers (e.g rayon cellulose, cotton cellulose, hemp and viscose) or combinations thereof. The nonwoven can be fanned by a number of different methods, including e.g. airlaid, wetlaid, spunbound or meltblown. The fibers can be carded (e.g. run through a comb) so that they are oriented in a particular direction. The webs can be bonded together in any manner including e.g hydroentangled, chemical bonded, needle punched or thermally bonded. In one embodiment, the nonwoven is comprised of a blend of polypropylene and PET fibers which are airlaid, carded and hydroentangled.

Any polymer film can be used. The polymer film can be selected from the group consisting of polyethylene, polypropylene, polyethylene copolymers, polypropylene copolymers, and PET.

The first and second substrates can be nonwoven. The nonwoven can have a basis weight of less than 40 grams per square meter (gsm), less than 35 gsm, less than about 30 gsrn, or even from about 10 gsm to about 40 gsm The nonwoven can be extensible to greater than 100% in the cross-web direction.

Various pre treatments (e.g. pleating and creping) and various post treatments, such as treatment with grooved rolls i.e. activation can be used to adjust the mechanical properties (e.g. extensibility) of the composite.

The adhesive can be applied to the first and or second substrate using a variety of application methods including slot, coating, non-contact coating, spraying including, e.g., spiral spraying and random spraying, screen printing, foaming, engraved roller, gravure roller, extrusion and meltblown.

The adhesive can be applied to one substrate.

Alternately, the adhesive can be applied to the first substrate and then contacted by the second substrate to form the composite. Pressure or tension can be used to aid in thrming the bonded composite. The composite can be formed within a disposable article manufacturing process. Alternatively, the composite is formed prior to the disposable article manufacturing process.

### Hot Melt Adhesive Composition

The adhesive composition is a hot melt adhesive. The adhesive can be a pressure sensitive adhesive. The adhesive composition can be light in color and can have good thermal stability. The adhesive can have an initial Gardner Color after manufacturing of less than about 3, or even less than about 2. Alternately, the adhesive can be pigmented to an opaque color such as e.g. pink, blue, white, etc.

The adhesive composition has low viscosity at application temperature. The viscosity is no greater than about 20,000 cps at around 177° C., no greater than about 15,000 cps at around 177° C., no greater than about 10,000 cps at around 177° C. no greater than about 7,000 cps at around 177° C., no greater than about 5,000 cps at around 177° C., from about 500 cps to about 20,000 cps at around 177° C., or even from about 1,000 cps to about 15,000 cps at around 177° C.

The adhesive composition gives Mechanical Hysteresis at 50% strain (coat weight 60 gsm) 2n^{d} cycle of no greater than 0.40, no greater than 0.38, no greater than 0.36, from about 0.05 to about 0.38 or even from about 0.10 to about 0.36.

The adhesive composition gives an Extension at 10 Newtons (N) of at least about 50%, at least about 100%, at least. about 150%, from about 50% to about 400%, or even from about 100% to about 300% and a Set of no greater than 50%, no greater than 40%, from about 5% to about 50%, or even from about 10% to about 35% when tested according to the 1-Peak Hysteresis Test Method (coat weight 60 gsm).

The adhesive composition can have a viscosity curve with a Viscosity Ratio (Viscosity @149° C. (cps)/Viscosity (q) 177° C.) that is no greater than about 7.5, no greater than about 6, no greater than about 5, or even no greater than about 4. A low viscosity ratio can indicate improved machining at high line speeds as the adhesive does not gain viscosity quickly as it cools.

The adhesive composition can include a tackifying resin with at least some aromatic content and a naphthenic oil.

In one embodiment, the styrene block copolymer, tackifying agent, the plasticizer and the wax make up at least 90% by weight, or even at least 95% by weight of the hot melt adhesive composition.

### Styrene Block Copolymer

The adhesive composition includes one or more styrene block copolymers having an unsaturated mid block.

The styrene block copolymer includes an aromatic vinyl polymer block and a conjugated diene polymer block. The blocks can be arranged in a variety of configurations including, e.g., linear, branched, radial, star and combinations thereof. The aromatic vinyl polymer block can be derived from a variety of aromatic vinyl compounds including, e.g., styrene, alpha-methylstyrene, beta-methylstyrene, o-, m-, p-methylstyrene, t-butylstyrene, 2,4,6-trimethylstyrene, monofluorostyrene, difluorostyrene, monochlorostyrene, dichlorostyrene, methoxystyrene, 1,3-vinylnaphthalene, vinylanthracene, indene, acenaphthylene, and combinations thereof. The diene polymer block can be derived from a variety of diene-containing compounds including, e.g., isoprene, butadiene, hexadiene, 2,3-dimethyl-1,3-butadiene and 1,3-pentadiene, and combinations thereof.

Useful styrene block copolymers include, e.g., triblock, multi-arm, and radial copolymers including, e.g., styrene-butadiene-styrene (SBS), styrene-isoprene-styrene (SIS), styrene-butadiene-isobutylene-styrene styrene-isoprene-butadiene-styrene (SIBS), and combinations thereof.

The one or more styrene block copolymers can have an average diblock content of no greater than about 10% by weight, no greater than about 5% by weight, no greater than about 3% by weight, or even no greater than about 1% by weight.

As an example, if a hot melt adhesive composition comprises two styrene block copolymers A and B. Polymer A is present at 25 weight % with a diblock content of 0% and polymer B is present at 25 weight % with a diblock content of 10%. The average diblock content of the one or more styrene block copolymers is calculated the following way: 0.5(0)+0.5(0.1)=0.05 or 5% by weight.

The one or more styrene block copolymers can have a styrene content of at least about 20% by weight, at least about 25% by weight, from about 20% to about 45% by weight, or even from about 25% to about 40% by weight.

The one or more styrene block copolymers can have an average Melt Flow Rate (MFR) ASTM D 1238 (200° C., 5.0 kg) of from about 20 to about 500, from about 50 to about 400, or even from about 150 to about 350.

The one or more styrene block copolymers are present at, at least 45% by weight, from about 45% by weight to about 75% by weight or even from about 50% by weight to about 70% by weight.

In one embodiment, at least one styrene block copolymer is present having a MFR ASTM 1238 (200° C., 5.0 kg) of at least about 50, at least about 100, at least about 150, at least about 200, from about 50 to about 500, from about 100 to about 450, or even from about 150 to about 400.

Useful commercially available styrene block copolymers include DPX 618, VECTOR 4411A and VECTOR 6241A, all available from TSRC Corporation (Plaquemine, La.).

Tackifying Agent

The adhesive includes a tackifying agent. The tackifying agent can be fluid or solid at room temperature. Suitable classes of tackifying agents include, e.g., aromatic, aliphatic and cycloaliphatic hydrocarbon resins, mixed aromatic and aliphatic modified hydrocarbon resins, aromatic modified aliphatic hydrocarbon resins, and hydrogenated versions thereof; terpenes, modified terpenes and hydrogenated versions thereof; natural rosins, modified rosins, rosin esters, and hydrogenated versions thereof; low molecular weight polylactic acid; and combinations thereof. Examples of useful natural and modified rosins include gum rosin, wood rosin, tall oil rosin, distilled rosin, hydrogenated rosin, dimerized rosin and polymerized rosin. Examples of useful rosin esters include e.g., glycerol esters of pale wood rosin, glycerol esters of hydrogenated rosin, glycerol esters of polymerized rosin, pentaerythritol esters of natural and modified rosins including pentaerythritol esters of pale wood rosin, pentaerythritol esters of hydrogenated rosin, pentaerythritol esters of tall oil rosin, and phenolic-modified pentaerythritol esters of rosin.

Useful tackifying agents are commercially available under a variety of trade designations including, e.g., the ESCOREZ series of trade designations from Exxon Mobil Chemical Company (Houston, Tex.) including ESCOREZ 5400 (1% aromatic content), ESCOREZ 5415, ESCOREZ 5600 (9.8% aromatic content), ESCOREZ 5690 (10% aromatic content), ESCOREZ 5615 (9.9% aromatic content), the EASTOTAC series of trade designations from Eastman Chemical (Kingsport, Tenn.) including EASTOTAC H-100R and EASTOTAC H-100L, and the WINGTACK series of trade designations from Cray Valley HSC (Exton, Pa.) including WINGTACK 86, WINGTACK EXTRA, and WINGTACK 95 and the PICCOTAC, KRISTALEX and PLASTOLYN series of trade designations from Eastman Chemical Company (Kingsport, Tenn.) including, e.g., PICCOTAC 8095, KRISTALEX 3100, PLASTOLYN 240 and PLASTOLYN 290.

The adhesive composition can include at least one tackifying agent with aromatic content. The tackifying agent can have an aromatic content of greater than 5%, greater than 20%, greater than 50%, from about 5% to about 20% by weight, from about 7.5% to about 15% by weight, or even from 5% by weight to less than 10% by weight. The aromatic content is measured by Nuclear Magnetic Resonance (NMR) spectroscopy.

The composition can include a tackifying agent with a softening point of less than 100° C., or even less than 95° C.

The adhesive composition can include less than 20% by weight, less than about 18% by weight, less than about 15% by weight, less than about 12% by weight, from about 2% to less than 20% by weight, from about 2% by weight to about 18%, from about 2% to about 15% by weight, or even from about 3% to less than 10% by weight of a tackifying agent.

### Plasticizer

The adhesive composition includes plasticizer. Suitable plasticizers include, e.g., oil (e.g. naphthenic oils, paraffinic oils (e.g., cycloparaffin oils), mineral oils), phthalate esters, adipate esters, olefin oligomers (e.g,, oligomers of polypropylene, polybutene, and hydrogenated polyisoprene), polybutenes, polyisoprene, hydrogenated polyisoprene, polybutadiene, benzoate esters, animal oil, plant oils castor oil, soybean oil high oleic soy oil)), derivatives of oils, glycerol esters of fatty acids, polyesters, polyethers, lactic acid derivatives and combinations thereof.

Useful commercially available plasticizers include CALSOL 5550, a naphthenic oil from Calumet Specialty Products Partners, LP (Indianapolis, Ind.), KAYDOL OIL mineral oil from Sonneborn (Tarrytown N.Y.) PARAPOL polybutene from Exxon Mobil Chemical Company (Houston, Tex.), OPPANOL polyisobutylene from BASF (Ludwigsjhafen, Germany), KRYSTOL 550 mineral oil from Petrochem Carless Limited (Surrey, England), PURETOL 35 and 15 both mineral oils from Petro Canada Lubricants Inc. (Mississauga, Ontario), PLENISH from Pioneer Dupont, and TPC 5230, polyisobutylene available from TPC Group (Houston, Tex.).

The plasticizer can be a naphthenic oil. Alternately, the plasticizer includes aromatic or naphthenic groups.

The plasticizer can be present in the adhesive composition in an amount of from about 10% to about 60% by weight, from about 15% to about 55% by weight, or even from about 15% to about 45% by weight.

Wax

The adhesive composition can include a wax. Useful classes of wax include, e.g., paraffin waxes, microcrystalline waxes, high density low molecular weight polyethylene waxes, by-product polyethylene waxes, polypropylene waxes, Fischer-Tropsch waxes, oxidized FisCher-Tropsch waxes, functionatized waxes such as acid, anhydride, and hydroxy modified waxes, animal waxes, vegetable waxes (e.g. soy wax) and combinations thereof. Useful waxes are solid at room temperature and preferably have a Ring and Ball softening point of from 50° C. to 170° C. Useful waxes are commercially available from a variety of suppliers including EPOLENE N and C series of trade designations from Westlake Chemical Corporation (Houston, Tex.) including e.g. EPOLENE N-21 and the LICOCENE series of trade designations from Clariant International Ltd. (Muttenz, Switzerland) including e.g. TP LICOCENE PP 6102.

The adhesive composition can include no greater than about 8.0% by weight, no greater than about 5% by weight, from about 1% by weight to about 7.5% by weight, or even from about 1% to about 5% by weight wax.

Additional Polymers

The adhesive composition optionally includes additional polymers (e.g. other styrene block copolymers, single-site (e.g. metallocene) catalyzed polyolefins, polyethylene homopolyrnerslcopolymers and polypropylene homopolymers/copolymers.) Useful additional polymers include VISTAMAXX 6102 (propylene/ethylene copolymer) available from ExxonMobil Chemical (Houston, Tex.).

### Additional Components

The adhesive composition optionally includes additional components including, e.g., stabilizers, antioxidants, adhesion promoters, ultraviolet light stabilizers, corrosion inhibitors, odor absorbers/neutralizers, colorants (e.g., pigments and dyes), fragrances, tillers (e.g. nano particles, clay, talc), surfactants, wetness indicators, superabsorbents, coextrusion coatings, processing aids and combinations thereof.

Useful antioxidants include, e.g., pentaerythritol tetrakis[3,(3,5-di-tert-butyl-4-hydroxyphenyl)propionate], 2,2′-methylene bis(4-methyl-6-tert-butylphenol), phosphites including, e.g., tris-(p-nonylphenyI)-phosphite (TNPP) and bis(2,4-di-tert-butylphenyl)4,4°-diphenylene-diphosphonite, di-stearyl-3,3′-thiodipropionate (DSTDP), and combinations thereof. Useful antioxidants are commercially available under a variety of trade designations including, e.g., the IRGANOX series of trade designations including, e.g., IRGANOX 1010, IRGANOX 565, and IRGANOX 1076 hindered phenolic antioxidants and IRGAFOS 168 phosphite antioxidant, all of which are available from BASF Corporation (Florham Park, N.J.), and ETHYL 702 4,4′-methylene bis(2,6-di-tert-butylphenol). When present, the adhesive composition preferably includes from about 0.1% by weight to about 2% by weight antioxidant,

### Disposable Article

The adhesive compositions and elastic composites of this invention can be incorporated into any suitable article including personal care garments, medical garments and industrial worker garments.

The elastic composite of this invention is useful in a variety of applications and constructions to improve comfort and fit including e.g., disposable absorbent articles including, e.g., diapers, training paints, swim wear, absorbent underpants, adult incontinence products, feminine hygiene products (e.g. sanitary napkins and liners), medical dressings (e.g., wound care products and bandages), surgical pads, medical gowns, caps, gloves, drapes, face masks, laboratory coats, coveralls, meat-packing products, furniture covers (e.g, car seat covers) and components of absorbent articles including, e.g., an absorbent element, absorbent cores, impermeable layers (e.g., backsheets), tissue (e.g., wrapping tissue), acquisition layers and woven and nonwoven web layers (e.g., top sheets, absorbent tissue).

The elastic composite of this invention is useful for elasticizing many areas of disposable articles including leg cuffs, waist portions, belly bands, side panels and fastening tabs/ears. The elastic composite of this invention can further be use to elasticize any portion of the disposable article or even the entire disposable article.

### EXAMPLES

### Test Procedures

Test procedures used in the examples and throughout the specification, unless stated otherwise, include the following.

### Viscosity Test Method

Viscosity is determined in accordance with ASTM D-3236 entitled, “Standard Test Method for Apparent viscosity of Adhesives and Coating Materials,” (Oct. 31, 1988), using a Brookfield Thermosel viscometer Model RVDV 2 and a number 27 spindle. The results are reported in centipoise (cps).

### Melt Flow Rate

Melt Flow Rate was run according to ASTM D 1238 at the conditions stated.

### Test Lamination Preparation

Test laminations were prepared by continuous slot coating the elastic adhesive composition between two nonwoven- substrates at an application temperature of 177° C., a nip pressure of 1.05 Newton/millimeter (N/mm) (6 pounds per linear inch (PLI)), and a run speed of at least 45.7 meters (m)/minute (min) (150 feet (ft)/min). Laminations were prepared with an adhesive coatweight of 60 and 75 grams per square meter (gsm) and the width of the adhesive was at least 7.6 centimeters (cm) (3 in). A sufficient amount of laminate is prepared such that at least 1.5 m (60 in) of representative lamination can be collected for testing.

In order to assist in getting peels started, flags were used. When a flag is put in a lamination it prevents the substrates from bonding to each other—giving an end by which to start the peel.

*For all examples, the nonwoven used is carded, hydroentangled and comprises 50/50 (PET/PP), it has a basis weigh: of 25 gsm and a cross direction tensile strength of 12 Newtons (N)/5 cm.

### Peel Force Test Method

Test laminates were prepared by coating adhesive between two substrates according to the Test Lamination Preparation method above. Peel force was determined using ASTM D1876-01 entitled; “Test Method for Determining Peel Resistance of Adhesive (T-Peel Test Method),” with the exception that the test was run at a rate of 30.5 cm (12 in) per min, instead of 25.4 cm (10 in) per ruin, over a period of 10 seconds, and 7 replicates are run instead of the 10 specified in ASTM D1876. The samples are run on an INSTRON type-test instrument. The test samples are 2.54 cm (1 in) in width and at least 10.2 cm (4 in) in length. The average peel force over 10 seconds of peeling is recorded, and the results are reported in grams. The initial peel force is measured at least 2.4 hours after the laminate is prepared.

### Mechanical Hysterisis

Test laminates were prepared by coating adhesive between two substrates according to the Test Lamination Preparation method. Test samples are prepared by cutting the laminates in cross-web direction, with 2.54 cm (1 in) in width and at least 7.6 cm (3 in) in length. Grip separation is set to 5.1 cm (2 in). The test is conducted at least 24 hours after the non woven laminate is prepared.

The strips prepared are extended to a certain strain (e.g. 50%, 100%, 150% or 200%) and then retracted to their original dimension. Subsequently the specimen goes through a second extension-retraction cycle with the same deformation. The cross head speed is set to 50.8 cm/min (20 in/min) during the extension portion of the cycle and 101.6 cm/min (40 in/min) during the retraction portion of the cycle. There is no holding time between extension and retraction. The samples are run on an INSTRON type-test instrument with at least 3 replicates. The energy loss during each hysteresis cycle and the total hysteresis cycle energy are recorded. The mechanical hysteresis is the ratio of the energy loss during the hysteresis cycle to the total hysteresis energy.

### 1—Peak Hysteresis Test Method

Test laminates were prepared by coating adhesive between two substrates according to the Test Lamination Preparation method.

Testing is performed using an INSTRON type-test instrument with a cross head speed of 51 cm/min (20 in/min), for both extension and retraction. The test specimen 5.1 cm (2 in) in width. Grip separation is also set to 5.1 cm (2 in). A sample test set normally consists of five replicates. The elastic is elongated by the grips until a force of 10 Newtons is reached. At that point the specimen is placed in a hold for 30 seconds and then allowed to retract to the point where the specimen returns to the 5.1 cm (2 in) initial gap setting. The data collected is the percent elongation, (mm/mm and/or percent) at which the specimen has been stressed to a force of 10 Newtons. During the 30 second hold, the force will diminish, and the magnitude of the loss in force is reported as force relaxation in Newtons (N). After the 30 second hold time has passed, the specimen is returned to the 5.1 cm (2 in) gap. The percent (%) set of the specimen is measured by the distortion of the two inch specimen length, determined by the % elongation of the specimen immediately after the cross head returns to the original gap as the force on the elastic approaches zero.

### Polymer Descriptions

POLYMER 1 is a <1 weight % diblock, SIS tri-block co-polymer containing 30% by weight styrene and having an ASTM 1) 1238 MFR (200° C., 5.0 kg)=303 g/10-min).

DPX 618 is a 10-15 weight % diblock, SIS radial triblock copolymer containing 31% by weight styrene and having an ASTM D 1238 MFR (200° C., 5.0 kg)=7 g/10-min).

Vector 4411A is a <1 weight % diblock, SIS tri-block co-polymer containing 44% by weight styrene and having an ASTM D 1238 of 40 g/10-min (200° C., 5.0 kg).

Vector 6241A is a <1 weight % diblock, SBS tri-block co-polymer containing 43% by weight styrene and having an ASTM D 1238 MFR of 23 g/10-min (200° C., 5.0 kg).

TABLE One

| | Ex 1 | Ex 2 | Ex 3 | Ex 4 | Ex 5 | Ex 6 | Ex 7 |
|---|---|---|---|---|---|---|---|
| VECTOR 4411 | 20 | 10 | | | | 15 | |
| VECTOR 6241 | 20 | 15 | 45 | | | | 10 |
| POLYMER 1 | 6 | 36 | | 50 | 70 | 44.5 | 49.5 |
| DPX 618 | 3 | | | | | | |
| Weight Avg | 63 | 191 | 23 | 303 | 303 | 237 | 256 |
| MFR ASTM D | | | | | | | |
| 1238 (200 C., | | | | | | | |
| 5.0 kg) of | | | | | | | |
| unsaturated | | | | | | | |
| SBC | | | | | | | |
| Weight Avg | 41 | 35 | 43 | 30 | 30 | 34 | 32 |
| styrene content | | | | | | | |
| of unsaturated | | | | | | | |
| SBC (weight %) | | | | | | | |
| Weight Avg | <1 | <1 | <1 | <1 | <1 | <1 | <1 |
| diblock content | | | | | | | |
| of unsaturated | | | | | | | |
| SBC (weight %) | | | | | | | |
| ESCOREZ 5690 | 13 | 10 | | 5 | 9.5 | 10 | 10 |
| PLASTOLYN | | | 3 | 4.5 | | | |
| 290 | | | | | | | |
| CALSOL 5550 | 35 | 26 | 52 | 40 | 20 | 30 | 30 |
| CALUMET | 2.5 | 2.5 | | | | | |
| FR6513 | | | | | | | |
| IRGANOX 1010 | 0.5 | 0.5 | 0.5 | 0.5 | 0.5 | 0.5 | 0.5 |

The Control is an SBC based stretchable hot melt adhesive that includes unsaturated styrene block copolymer and 28% tackifier.

TABLE Two

| Viscosity (cps) | Control | Ex 1 | Ex 2 | Ex 3 | Ex 4 | Ex 5 | Ex 6 | Ex 7 |
|---|---|---|---|---|---|---|---|---|
| @149° C. (300° F.) | 26100 | 28000 | 60200 | 21000 | 20900 | 44950 | 41400 | 60200 |
| @163° C. (325° F.) | 13200 | 12200 | 27200 | 10900 | 8450 | 19400 | 16550 | 27200 |
| @177° C. (350° F.) | 7225 | 6350 | 13780 | 5900 | 5250 | 10900 | 8900 | 7813 |
| Viscosity Ratio | 3.6 | 4.4 | 4.4 | 3.6 | 4.0 | 4.1 | 4.7 | 7.7 |
| @149° C./177° C. | | | | | | | | |

TABLE Three

| | | | | | | | |
|---|---|---|---|---|---|---|---|
| Control | Ex 1 | Ex 2 | Ex 3 | Ex 4 | Ex : | Ex 6 | Ex 7 |

Mechanical Hysteresis (coat weight 60 gsm)

| | | | | | | | | |
|---|---|---|---|---|---|---|---|---|
| 50% strain | | | | | | | | |
| 1^{st} cycle | 0.597 | 0.533 | 0.500 | 0.494 | 0.493 | 0.480 | 0.502 | 0.509 |
| 2^{nd} cycle | 0.405 | 0.356 | 0.321 | 0.337 | 0.334 | 0.300 | 0.331 | 0.326 |
| 100% strain | | | | | | | | |
| 1^{st} cycle | 0.745 | 0.694 | 0.651 | 0.675 | 0.682 | 0.620 | 0.647 | 0.673 |
| 2^{nd} cycle | 0.510 | 0.453 | 0.410 | 0.457 | 0.462 | 0.387 | 0.420 | 0.428 |

1-Peak Hysteresis Testing (coat weight 60 gsm)

| | | | | | | | | |
|---|---|---|---|---|---|---|---|---|
| Extension | 165 | 275 | 181 | 189 | 171 | 184 | 199 | 188 |
| at 10N (%) | | | | | | | | |
| Set (%) | 27 | 39 | 24 | 47 | 44 | 22 | 32 | 35 |

Adhesion (coat weight 60 gsm)

| | | | | | | | | |
|---|---|---|---|---|---|---|---|---|
| Average peel | 845 | 442 | 277 | 394 | 520 | 336 | 501 | 520 |
| (gram force) | | | | | | | | |

Mechanical Hysteresis (coat weight 75 gsm)

| | | | | | | | | |
|---|---|---|---|---|---|---|---|---|
| 50% hysteresis | | | | | | | | |
| 1st cycle | 0.579 | 0.516 | 0.495 | 0.478 | 0.496 | 0.485 | 0.483 | 0.493 |
| 2nd cycle | 0.387 | 0.324 | 0.307 | 0.308 | 0.325 | 0.298 | 0.302 | 0.295 |
| 100% hysteresis | | | | | | | | |
| 1st cycle | 0.726 | 0.690 | 0.659 | 0.670 | 0.677 | 0.627 | 0.663 | 0.651 |
| 2nd cycle | 0.487 | 0.443 | 0.416 | 0.442 | 0.448 | 0.381 | 0.422 | 0.404 |

1-Peak Hysteresis Testing (coat weight 75 gsm)

| | | | | | | | | |
|---|---|---|---|---|---|---|---|---|
| Extension at | 124 | 145 | 143 | 165 | 140 | 128 | 119 | 119 |
| 10N (%) | | | | | | | | |
| Set (%) | 18 | 22 | 16 | 34 | 29 | 14 | 16 | 18 |

What is claimed is

1. An elastic composite comprising:
a first and second substrate; and
a hot melt adhesive composition between the first and second substrates thereby bonding the first and second substrates to each other and providing elasticity in the bonded area, said hot melt adhesive composition comprising:
from about 45% to about 75% by weight of one or more styrene block copolymers having an unsaturated mid-block and an average styrene content of at least about 20%,
less than 20% by weight of a tackifying agent,
from about 10% to about 60% by weight of a plasticizer,
the hot melt adhesive composition having a viscosity of no greater than about 20,000 cps at 177° C.

2. The elastic composite of claim 1 wherein the hot melt adhesive composition has a Mechanical Hysteresis 50% strain (coat weight 60 gsm) 2nd cycle of no greater than 0.38.

3. The elastic composite of claim 1 wherein the one or more styrene block copolymers has an average diblock content of no greater than about 10% by weight.

4. The elastic composite of claim 1 wherein the one or more styrene block copolymers has an average styrene content of at least about 25% by weight.

5. The elastic composite of claim 1 wherein the hot melt adhesive composition comprises from about 2% to about 15% by weight of a tackifying agent.

6. The elastic composite of claim 1 wherein the hot melt adhesive composition comprises from about 3% to less than 10% by weight of a tackifying agent. 7, The elastic composite of claim 1 wherein at least one styrene block copolymer is present having an ASTM D 1238 (200° C., 5.0 kg) melt flow rate of at least about 50 g/10 minutes.

8. The elastic composite of claim 1 wherein the hot melt adhesive composition further comprises a paraffin wax.

9. The elastic composite of claim 1 wherein the first and second substrate are nonwoven.

10. The elastic composite of claim 9 wherein the nonwoven is carded and manufactured in a method selected from hydroentangled and thermally bonded.

11. The elastic composite of claim 9 wherein the nonwoven is extensible to greater than 100% in the cross web direction.

12. The elastic composite of claim 1 wherein the hot melt adhesive is applied to the substrate using an applicator method selected from the group consisting of slot and non-contact coating.

13. The elastic composite of claim 1 wherein the hot melt adhesive is applied to the substrate using an applicator method selected from the group consisting of screen printing, spraying, comb shim slot and gravure roll.

14. A disposable article comprising the elastic composite of claim 1.

15. The disposable article of claim 14 wherein the elastic composite is used in an application selected from the group consisting of ear, waist band, belly band and side panel.

16. The disposable article of claim 14 selected from the group consisting of diaper, adult incontinence product, feminine hygiene product and medical bandage.

17. A hot melt adhesive composition that can be used to create a bonded, elasticized region, comprising:
from about 45% to about 75% by weight of one or more styrene block copolymers, the styrene block copolymers having unsaturated backbones, an average styrene content of at least about 25 ⁰% by weight, and a diblock content of no greater than about 10% by weight,
less than 20% by weight of a tackifying agent and,
from about 15% to about 45% by weight plasticizer, the adhesive composition having a viscosity of no greater than about 20,000 cps at 177° C.

18. The hot melt adhesive of claim 17 wherein the hot melt adhesive composition has a Mechanical Hysteresis 50% strain (coat weight 60 gsm) 2nd cycle of no greater than 0.38.

19. The hot melt adhesive composition of claim 17 with a viscosity of no greater than about 15,000 cps at 177° C.

20. The hot melt adhesive composition of claim 17 wherein at least one styrene block copolymer is present having a ASTM D 1238 (200° C., 5.0 kg) melt flow rate of at least about 50.
